(19) United States
(12) Patent Application Publication
(10) Pub. No.: US 2026/0097017 A1
(43) Pub. Date: Apr. 9, 2026

(54) TASIPIMIDINE AND CYP2D6 INHIBITOR COMBINATION TREATMENT

(71) Applicant: ORION CORPORATION, Espoo (FI)

(72) Inventors: Marja KÄHKÖNEN, Espoo (FI); Juha ROURU, Espoo (FI); Päivi TAAVITSAINEN, Espoo (FI); Johanna TUUNAINEN, Espoo (FI)

(21) Appl. No.: 19/115,955

(22) PCT Filed: Sep. 27, 2023
(86) PCT No.: PCT/FI2023/050545

§ 371 Date: Mar. 27, 2025

(30) Foreign Application Priority Data
Sep. 28, 2022 (FI) 20225853

(51) Int. Cl. A61K 31/4178; A61K 31/137; A61K 31/138; A61K 31/36; A61K 31/4433; A61K 31/451; A61K 31/4525; A61K 31/49; A61K 31/517

(52) U.S. Cl. CPC A61K 31/4178; A61K 31/137; A61K 31/138; A61K 31/36; A61K 31/4433; A61K 31/451; A61K 31/4525; A61K 31/49; A61K 31/517

(57) ABSTRACT

The invention relates to co-administration of a CYP2D6 inhibitor with tasipimidine, or a pharmaceutically acceptable salt thereof, in order to improve the pharmacokinetic profile of tasipimidine by decreasing its clearance and inter-individual variation. The invention also relates to a combination of tasipimidine, or a pharmaceutically acceptable salt thereof, and a CYP2D6 inhibitor, and the use of such a combination for the treatment of a disorder, condition or disease where an alpha2A agonist is indicated to be useful, for example, for use in the treatment of neuropsychiatric disorders.

### TECHNICAL FIELD

The present disclosure relates to the use of tasipimidine, or a pharmaceutically acceptable salt thereof, in combination with a CYP2D6 inhibitor in order to improve the pharmacokinetic profile of tasipimidine by decreasing its clearance and inter-individual variation.

### BACKGROUND OF THE INVENTION

Alpha-2 adrenoceptor agonists have been in clinical use since the mid-1960s when clonidine was introduced as an antihypertensive drug. Alpha-2 adrenoceptor activation is known to result in a variety of responses from several organs and tissues. Activation of presynaptic alpha-2 adrenoceptors located in sympathetic nerve endings inhibits the release of the neurotransmitter noradrenaline. Activation of postsynaptic alpha-2 adrenoceptors in the central nervous system leads to inhibition of sympathetic activity, causing decreases in blood pressure and heart rate, decreased arousal, sedation and relief of anxiety. Activation of alpha-2 adrenoceptors at the spinal level results in analgesia. Peripheral alpha-2 adrenoceptors in blood vessels mediate vascular smooth muscle contraction. There are three distinct subtypes of alpha-2 adrenoceptors, alpha-2A, alpha-2B and alpha-2C, each encoded by their own gene. According to the current knowledge, the major part of the alpha-2 adrenergic actions is mediated by the alpha-2A subtype. The other subtypes act as “fine-tuners” of related functions and may sometimes have opposite effects. Some evidence suggests also that stimulation of the vascular alpha-2B receptors is responsible for the transient vasoconstriction seen after alpha-2 agonist administration.

Currently available, centrally-acting alpha2 agonists are indicated for the treatment of hypertension (clonidine), spasticity (tizanidine), attention deficit hyperactivity disorder (guanfacine), intensive care sedation and procedural sedation (dexmedetomidine). At sufficiently high dose levels they produce a reduction in blood pressure and heart rate and sedation that are the intended therapeutic effects for some of the compounds, and as adverse effects dry mouth, dizziness, high blood pressure at higher doses, and rarer effects such as atrioventricular conduction block or dissociation particularly in situations with high parasympathetic tone.

Tasipimidine is the International Nonproprietary Name (INN) of the compound 2-(5-methoxyisochroman-1-yl)-4,5-dihydro-1H-imidazole represented by the following structural formula (I):

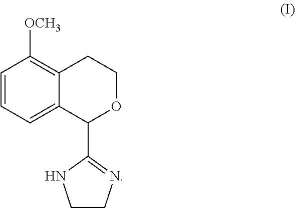 [SMILES: COc1cccc2c1CCOC2C1=NCCN1]

Tasipimidine is a novel, orally active, highly selective alpha2A adrenoceptor agonist. Its high oral bioavailability and alpha2A selectivity differentiate it from dexmedetomidine, the currently approved and most specific alpha2 adrenoceptor agonist. In addition, tasipimidine has a shorter elimination half-life (t1/2) than clonidine, (clonidine t1/2=14 h), faster onset of action, and is more sedative. In dogs tasipimidine has shown to be effective in relieving situational anxiety and fear triggered by noise or owner departure.

Tasipimidine and its pharmaceutically acceptable salts have been disclosed in WO 2013/150173, which also lists some possible indications for tasipimidine. Tasipimidine and salts thereof, particularly sulfate salt, may be prepared using the method described, for example, in WO 2019/106238. In addition to indications mentioned above for alpha2 agonists, tasipimidine has several potential indications with good confidence on efficacy and high unmet need, like agitation in dementia, panic disorder, social anxiety disorder/agoraphobia, insomnia, MDD (major depressive disorder) with anxious distress.

The therapeutic marginal of alpha 2 agonists is known to be relatively narrow especially during chronic use, and it depends on target population and indication. The cardiovascular effects, like orthostatic hypotension, occur at plasma concentrations and exposures close to those mediating therapeutic central nervous system effects.

The cytochrome P450 family 2 subfamily D member 6 (CYP2D6) is a genetically polymorphic drug-metabolizing enzyme. The new finding demonstrating that tasipimidine is metabolized by CYP2D6 enzyme together with new data on human pharmacokinetics demonstrates relatively large inter-individual variation. This is due to known genetic polymorphism of the CYP2D6. Hence, certain individuals will eliminate tasipimidine quickly (ultra-rapid metabolizers) while others slowly (poor metabolizers), and the rest will eliminate tasipimidine at a rate somewhere in between ultra-rapid and poor metabolizers (intermediate and normal metabolizers). Tasipimidine's elimination half life in humans is relatively short leading to high fluctuation in peak and through concentrations if the compound is dosed one to three times daily. This kind of large inter-individual variation and short elimination half life may be difficult to handle in clinical practice for a compound with relative narrow therapeutic marginal.

### SUMMARY OF THE INVENTION

It has now been found that use of tasipimidine, or a pharmaceutically acceptable salt thereof, in combination with a CYP2D6 inhibitor reduces the inter-individual variation of tasipimidine pharmacokinetics and prolong the elimination half life making the product easier to use in clinical practice, i.e. co-administration of a CYP2D6 inhibitor converts all types of metabolizers to a poor metabolizer phenotype, so there is no need to genotype the patients before starting the treatment with tasipimidine and less frequent dosing regimen can be used without the loss of therapeutic efficacy.

If a drug is metabolized too quickly, it may decrease the drug's efficacy while if the drug is metabolized too slowly, side effects may result. This may result if same fixed dose of tasipimidine, a CYP2D6 substrate, is administered to patients without knowing their CYP2D6 genotype. Therefore, combining tasipimidine with a CYP2D6 inhibitor and by that eliminating the inter-individual variability in CYP2D6 mediated metabolism enhances the efficacy, and on the other hand, reduces the potential for an adverse event.

There are several known inhibitors of CYP2D6, with classification of strong, moderate, and weak or mild inhibitors. Examples of strong CYP2D6 inhibitors include, but are not limited to, paroxetine, bupropion, fluoxetine, quinidine, terbinafine, dacomitinib, pridopidine, tipranavir, and 3,4-methylenedioxymethamphetamine (MDMA, ecstasy). Examples of moderate inhibitors include, but are not limited to, abiraterone, cinacalcet, darifenacin, darunavir, duloxetine, givosiran, lorcaserin, mirabegron, perhexiline, rolapitant, and thioridazine. Examples of weak inhibitors include, but are not limited to, amiodarone, celecoxib, cimetidine, clobazam, cobicistat, escitalopram, fluvoxamine, labetalol, ritonavir, sertraline, and vemurafenib. These classifications are based upon US Food and Drug Administration (FDA) and Washington University Drug Interaction Database guidance. Other sources may use a different classification system resulting in some agents being classified differently.

The foregoing as well as other feature and advantages of the present teachings will be more fully understood from the following description and claims.

### BRIEF DESCRIPTION OF THE DRAWINGS

FIG. 1 shows the correlation between CYP2D6 activity score and dose corrected AUCinf and Cmax of tasipimidine.

FIG. 2 shows individual tasipimidine plasma concentration profiles after 10 μg oral dose with and without paroxetine co-administration.

### DETAILED DESCRIPTION OF THE INVENTION

The present disclosure relates to combination of tasipimidine, or a pharmaceutically acceptable salt thereof, and a CYP2D6 inhibitor. In particular, the present disclosure relates to a therapeutic use and method of treatment wherein tasipimidine, or a pharmaceutically acceptable salt thereof, and a CYP2D6 inhibitor are administered in combination to a human being. In addition, the present disclosure relates to such a combination for use in the treatment of neuropsychiatric disorders in a human being in need thereof.

Accordingly, in one embodiment, the present disclosure relates to tasipimidine, or a pharmaceutically acceptable salt thereof, in combination with a CYP2D6 inhibitor for use in the treatment of disorder, condition or disease where an alpha2A agonist is indicated to be useful, for example, for use in the treatment of neuropsychiatric disorders, such as insomnia, agitation, aggression, anxiety, depression, and panic disorder.

It should be noted that said therapeutic use or method for treating a human being is intended to encompass all of the potential uses of tasipimidine, including all potential uses which derive from tasipimidine's activity as alpha2A adrenoceptor agonist e.g. its use as hypotensive agent, anxiolytic, analgesic, sedative, and the like. The combination according to the present disclosure is preferably useful in treating insomnia disorder or insomnia disorder with comorbid depression, anxiety or pain. In addition, it is particularly useful in treating anxiety, agitation or depression. It is especially useful in treating anxiety or agitation or aggression in patients with dementia, e.g. Alzheimer's disease. Agitation may be chronic or acute agitation. It is specifically useful in treating agitation associated with neurodegenerative conditions selected from the group consisting of: Alzheimer disease, frontotemporal dementia, dementia, dementia with Lewy bodies, post-traumatic stress disorder, Parkinson's disease, vascular dementia, vascular cognitive impairment, Huntington's disease, multiple sclerosis, Creutzfeldt-Jakob disease, multiple system atrophy, and progressive supranuclear palsy, senile dementia of the Alzheimer type; or agitation associated with neuropsychiatric conditions selected from the group consisting of: schizophrenia, bipolar disorder, bipolar mania, delirium, and depression, including dementia or mood disorders in subjects with major depression (e.g. stress-related major depression); or agitation associated with other conditions such as OPD/IPD procedures (e.g. MRI, CT or CAT scan, lumbar puncture, bone marrow aspiration/biopsy, tooth extraction and other dental procedures); or agitation associated with alcohol, opioid use disorder, opioid withdrawal and substance abuse withdrawal. Further, it is useful in treating delirium, hyperactive delirium, benzodiazepine or alcohol or opioid or tobacco withdrawal, premature ejaculation, tachycardia, restless leg syndrome, hot flashes, post traumatic stress disorder, panic disorder, pain, chronic pelvic pain syndrome, breakthrough cancer pain, traumatic brain injury, tardive dyskinesia, social anxiety disorder, agoraphobia, and attention deficit hyperactivity disorder (ADHD).

In one embodiment, the present disclosure relates to tasipimidine, or a pharmaceutically acceptable salt thereof, for use in combination with a CYP2D6 inhibitor.

In one embodiment, the present disclosure relates to co-administration of tasipimidine, or a pharmaceutically acceptable salt thereof, and a CYP2D6 inhibitor, for use to reduce the inter-individual variability in CYP2D6 mediated metabolism in human beings in need of treatment with tasipimidine.

In one embodiment, the present disclosure relates to the combination of tasipimidine, or a pharmaceutically acceptable salt thereof, and a CYP2D6 inhibitor for use to increase tasipimidine plasma levels in a human being in need of treatment with tasipimidine.

In one embodiment, the present disclosure relates to the combination of tasipimidine, or a pharmaceutically acceptable salt thereof, and a CYP2D6 inhibitor for use to prolong the elimination half life of tasipimidine in a human being in need of treatment with tasipimidine.

In one embodiment, the present disclosure relates to a CYP2D6 inhibitor for use to inhibit the metabolism of tasipimidine, wherein tasipimidine, or a pharmaceutically acceptable salt thereof, is present in the body of the human being at the same time as a CYP2D6 inhibitor.

In one embodiment, the present disclosure relates to the administration of a CYP2D6 inhibitor to a human being in need of treatment with tasipimidine, for use to increase the metabolic lifetime of tasipimidine, wherein tasipimidine, or a pharmaceutically acceptable salt thereof, is present in the body of the human being at the same time as a CYP2D6 inhibitor.

In one embodiment, the present disclosure relates to a CYP2D6 inhibitor for use to correct ultra-rapid metabolism of tasipimidine in a human being in need thereof.

In one embodiment, the present disclosure relates to a CY2D6 inhibitor administered in conjunction with tasipimidine, or a pharmaceutically acceptable salt thereof, for use to improve the therapeutic properties of tasipimidine in treating neuropsychiatric disorder of a human being in need of treatment for a neuropsychiatric disorder.

In one embodiment, the present disclosure relates to a combination of tasipimidine, or a pharmaceutically acceptable salt thereof, and a CYP2D6 inhibitor for use in the treatment of a neuropsychiatric disorder in a human being in need thereof.

In one embodiment, the present disclosure relates to a combination of tasipimidine, or a pharmaceutically acceptable salt thereof, and a CYP2D6 inhibitor for use to reduce an adverse event associated with treatment by tasipimidine, in a human being in need of tasipimidine treatment, wherein the human being is at risk of experiencing the adverse event as a result being treated with tasipimidine.

In one embodiment, the present disclosure relates to a CYP2D6 inhibitor for use to improve the pharmacokinetic profile of tasipimidine.

In one embodiment, the present disclosure relates to the use of a combination of tasipimidine, or a pharmaceutically acceptable salt thereof, and a CYP2D6 inhibitor in the manufacture of a medicament for the treatment of neuropsychiatric disorders.

In one embodiment, the present disclosure relates to a method of administering tasipimidine, or a pharmaceutically acceptable salt thereof, in combination with a CYP2D6 inhibitor to a human being in need of treatment with tasipimidine.

In one embodiment, the present disclosure relates to a method of reducing the inter-individual variability in CYP2D6 mediated metabolism in human beings in need of treatment with tasipimidine, comprising co-administering a CYP2D6 inhibitor with tasipimidine, or a pharmaceutically acceptable salt thereof, to the human being.

In one embodiment, the present disclosure relates to a method of increasing tasipimidine plasma levels in a human being in need of treatment with tasipimidine, comprising co-administering a CYP2D6 inhibitor with tasipimidine, or a pharmaceutically acceptable salt thereof, to the human being.

In one embodiment, the present disclosure relates to a method of prolonging the elimination half life of tasipimidine in a human being in need of treatment with tasipimidine, comprising co-administering a CYP2D6 inhibitor with tasipimidine, or a pharmaceutically acceptable salt thereof, to the human being.

In one embodiment, the present disclosure relates to a method of inhibiting the metabolism of tasipimidine, comprising administering a CYP2D6 inhibitor to a human being, and wherein tasipimidine, or a pharmaceutically acceptable salt thereof, is present in the body of the human being at the same time as a CYP2D6 inhibitor.

In one embodiment the present disclosure relates to a method of increasing the metabolic lifetime of tasipimidine, comprising administering a CYP2D6 inhibitor to a human being in need of treatment with tasipimidine, and wherein tasipimidine, or a pharmaceutically acceptable salt thereof, is present in the body of the human being at the same time as a CYP2D6 inhibitor.

In one embodiment the present disclosure relates to a method of correcting ultra-rapid metabolism of tasipimidine, comprising administering a CYP2D6 inhibitor to a human being in need thereof, such as a human being in need of treatment of a neuropsychiatric disorder.

In one embodiment, the present disclosure relates to a method of improving the therapeutic properties of tasipimidine, in treating neuropsychiatric disorders comprising administering a CY2D6 inhibitor in conjunction with administration of tasipimidine, or a pharmaceutically acceptable salt thereof, to a human being in need of treatment for a neuropsychiatric disorder.

In one embodiment, the present disclosure relates to a method of reducing an adverse event associated with treatment by tasipimidine, comprising administering tasipimidine, or a pharmaceutically acceptable salt thereof, in combination with a CYP2D6 inhibitor to a human being in need of tasipimidine treatment, wherein the human being is at risk of experiencing the adverse event as a result being treated with tasipimidine.

In one embodiment, the present disclosure relates to a method for the treatment of a neuropsychiatric disorder, which method comprises administering to a human being in need of such treatment tasipimidine, or a pharmaceutically acceptable salt thereof, in combination with a CYP2D6 inhibitor.

Tasipimidine, or a pharmaceutically acceptable salt thereof, and a CYP2D6 inhibitor compound may be administered in separate compositions or dosage forms, or may be administered in a single composition or dosage form comprising both. Additionally, the two compounds may be administered at the same time, but this is not required. The compounds can be given at different timed as long as both are in the body of the human being at the same time for at least a portion of the time that treatment by co-administration is being carried out.

In one embodiment, the present disclosure relates to combination therapy wherein tasipimidine, or a pharmaceutically acceptable salt thereof, and a CYP2D6 inhibitor are administered together, as part of the same pharmaceutical composition.

In one embodiment, the present disclosure relates to combination therapy wherein tasipimidine, or a pharmaceutically acceptable salt thereof, and a CYP2D6 inhibitor are administered simultaneously, in two separate pharmaceutical compositions.

In one embodiment, the present disclosure relates to combination therapy wherein tasipimidine, or a pharmaceutically acceptable salt thereof, and a CYP2D6 inhibitor are administered separately as part of an appropriate dosage regimen designed to obtain the benefits of the combination therapy. The appropriate dosage regimen, the amount of each dose administered, and specific intervals between doses of each active agent will depend on the person being treated, and the source and severity of the condition.

In one embodiment, the present disclosure relates to combination therapy wherein tasipimidine, or a pharmaceutically acceptable salt thereof, and the CYP2D6 inhibitor are administered sequentially as part of an appropriate dosage regimen, i.e. the delay in administering the second component should be such that all agents are present in the body of the human being so as to produce synergistic effect of the combination. The appropriate dosage regimen, the amount of each dose administered, and specific intervals between doses of each active agent will depend on the person being treated, and the source and severity of the condition.

All usual administration routes are suitable for administering the combination of the present disclosure. When administered separately or sequentially, administration can be via alternative routes.

The therapeutic dose to be given to a human being in need of the treatment will vary depending on the combination being administered, the species, the age and the sex of the person being treated, the particular condition being treated, as well as the route and method of administration. The suitable amount of a CYP2D6 inhibitor is an amount sufficient to block tasipimidine metabolism, and a suitable amount of tasipimidine, or a pharmaceutically acceptable salt thereof, is an amount sufficient to treat a neuropsychiatric disorder in question. In one embodiment, tasipimidine, or a pharmaceutically acceptable salt thereof, is administered for the treatment of a neuropsychiatric disorder, in an amount not more than 600 μg, generally not more than 300 μg, preferably not more than 150 μg, for example in an amount ranging from about 5 μg to about 600 μg, typically from about 5 μg to about 300 μg, preferably from about 5 μg to about 200 μg, for example from about 10 μg to about 150 μg per day to a patient. The dose can be administered once daily or divided to several times a day, for example twice daily or three times a day.

Any CYP2D6 inhibitor may be used in combination with tasipimidine, or a pharmaceutically acceptable salt thereof. Examples of CYP2D6 inhibitor compounds that can be used in combination with tasipimidine, or a pharmaceutically acceptable salt thereof, according to the present disclosure, include, but are not limited to, paroxetine, bupropion, fluoxetine, quinidine, terbinafine, dacomitinib, pridopidine, tipranavir, 3,4-methylenedioxymethamphetamine, abiraterone, cinacalcet, darifenacin, darunavir, duloxetine, givosiran, lorcaserin, mirabegron, perhexiline, rolapitant, thioridazine, amiodarone, celecoxib, cimetidine, clobazam, cobicistat, escitalopram, fluvoxamine, labetalol, ritonavir, sertraline, and vemurafenib, and their pharmaceutically acceptable salts, esters and prodrugs. A CYP2D6 inhibitor according to the present disclosure is preferably a strong CYP2D6 inhibitor, for example, paroxetine, bupropion, fluoxetine, quinidine, terbinafine, dacomitinib, pridopidine, tipranavir, or 3,4-methylenedioxymethamphetamine; such as paroxetine or bupropion. In addition to the compounds listed above, there are other compounds which may be effective in enhancing the delivery of tasipimidine by inhibiting the CYP2D6 enzyme.

In one embodiment, the present disclosure relates to a combination comprising (i) tasipimidine, or a pharmaceutically acceptable salt thereof, and (ii) a CYP2D6 inhibitor.

In one embodiment, the present disclosure relates to a combination comprising tasipimidine, or a pharmaceutically acceptable salt thereof, and a CYP2D6 inhibitor selected from paroxetine, bupropion, fluoxetine, quinidine, terbinafine, dacomitinib, pridopidine, tipranavir, and 3,4-methylenedioxymethamphetamine, or their pharmaceutically acceptable salts. For example, the present disclosure relates to the combination comprising tasipimidine, or a pharmaceutically acceptable salt thereof, and paroxetine, bupropion, fluoxetine, quinidine or terbinafine, or their pharmaceutically acceptable salts. Preferably, the present disclosure relates to the combination comprising tasipimidine, or a pharmaceutically acceptable salt thereof, and paroxetine, bupropion or fluoxetine, or their pharmaceutically acceptable salts. Even more preferably, the present disclosure relates to the combination comprising tasipimidine, or a pharmaceutically acceptable salt thereof, and paroxetine or bupropion, or a pharmaceutically acceptable salt thereof; such as the combination comprising tasipimidine, or a pharmaceutically acceptable salt thereof, and paroxetine, or a pharmaceutically acceptable salt thereof.

In one embodiment, the present disclosure relates to a pharmaceutical composition comprising (i) tasipimidine, or a pharmaceutically acceptable salt thereof, (ii) a CYP2D6 inhibitor, and (iii) one ore more pharmaceutically acceptable excipients and/or carriers.

In one embodiment, the present disclosure relates to a pharmaceutical composition for combination therapy involving administration of tasipimidine, or a pharmaceutically acceptable salt thereof, and a CYP2D6 inhibitor together or separately, comprising (i) a therapeutically effective amount of tasipimidine, or a pharmaceutically acceptable salt thereof; (ii) a CYP2D6 inhibitor; and (iii) one ore more pharmaceutically acceptable excipients and/or carriers.

The abovementioned pharmaceutical composition may be prepared by commonly know manufacturing methods, e.g. by mixing the active ingredients with the conventional excipient(s) and/or carrier(s) that are well known in the art; such as fillers, binders, diluents, disintegrating agents, lubricants, solvents, gel forming agents, emulsifiers, stabilizers, colorants, and/or preservatives. The pharmaceutical composition may be, for example, tablet, capsule, granule, suppository, emulsion, suspension, or solution. Depending on the route of administration and the galenic form, the amount of the active ingredient in a formulation can typically vary between 0.01% and 100% by weight.

The terms used herein have the meanings indicated below.

The term “tasipimidine”, as used herein, refers to 2-(5-methoxyisochroman-1-yl)-4,5-dihydro-1H-imidazole in free form and to pharmaceutically acceptable salts thereof, particularly a sulfate salt.

The term “CYP2D6 inhibitor”, as used herein, refers to a drug that reduces the activity of a CYP2D6 enzyme.

The term “strong CYP2D6 inhibitor”, as used herein, refers to a drug that causes at least a 5-fold increase in the plasma AUC values of sensitive substrates metabolized through CYP2D6, or more than 80% in decrease in clearance thereof.

The term “moderate CYP2D6 inhibitor”, as used herein, refers to a drug that causes at least a 2-fold increase in the plasma AUC values of sensitive substrates metabolized through CYP2D6, or 50-80% in decrease in clearance thereof.

The term “weak CYP2D6 inhibitor”, as used herein, refers to a drug that causes at least a 1.25-fold but less than 2-fold increase in the plasma AUC values of sensitive substrates metabolized through CYP2D6, or 20-50% in decrease in clearance thereof.

The “pharmaceutically acceptable salts”, according to the present disclosure include therapeutically active, non-toxic base and acid salt forms, which tasipimidine or a CYP2D6 inhibitor is able to form with both organic and inorganic bases and acids.

The term “neuropsychiatric disorders”, as used herein, refers to disorders or conditions that are accompanied by challenges with sleep, eating, learning, language development and motor skills, and by mood swings, anxiety or compulsiveness, and sensory processing sensitivity. Examples of neuropsychiatric disorders include, but are not limited to, sleep disorders (for example insomnia with or without comorbidities), affective (mood) disorders (for example, depression), psychiatric disorders (for example, eating disorders, addictions, agitation, anxiety, or psychosis), cerebral function disorders, movement disorders, degenerative diseases (for example dementias), traumatic brain injury, chronic traumatic encephalopathy, neurotic disorders (for example, post-traumatic stress disorder (PTSD)), motor neuron diseases, neurodegenerative diseases, seizure disorders, and headaches.

The term “Cmax”, as used herein, refers to the highest concentration of a drug in the blood plasma after a dose is given.

The term “AUCinf”, as used herein, refers to the definite integral of the concentration of a drug in blood plasma as a function of time.

The term “t1/2”, as used herein, refers to the elimination half-life.

The present disclosure will be explained in more detailed by the following examples. The examples are meant for illustrating purposes only and do not limit the scope of the invention defined in the claims.

### Example 1: In Vitro Studies

The objective of these studies was to identify enzymes involved in the metabolism of tasipimidine in human. The test substance tasipimidine and the reference compounds for main tasipimidine metabolites 1-(4,5-dihydro-1H-imidazol-2-yl) isochroman-5-ol (metabolite 1), 2-(5-methoxyisochroman-1-yl)-1H-imidazole (metabolite 2), and 1-(1H-imidazol-2-yl) isochroman-5-ol (metabolite 3) were supplied by Orion Pharma. Tasipimidine was incubated with the recombinant human (rh) cytochrome P450 (CYP) enzymes listed in the Table 1. To further support the findings of recombinant enzyme incubations tasipimidine was incubated with human hepatocytes with two enzyme inhibitors, i.e. 1-aminobenzotriazole (ABT, non-selective CYP inhibitor), and quinidine (CYP2D6 inhibitor).

Moreover, enzyme kinetics of tasipimidine metabolism was studied in rhCYP2D6.

TABLE 1
In vitro studies conducted to identify enzymes metabolizing tasipimidine

| Human enzyme | Enzyme(s) of | | |
|---|---|---|---|
| preparate | interest | Used inhibitor(s) | Purpose |
| rhCYP | 1A1, 1A2, 2A6, | — | Study which CYP involved |
| Supersomes^{1, 2} | 2B6, 2C8, 2C9, | | in metabolism |
| | 2C19, 2D6, 2E1, | | |
| | 3A4, 3A5 | | |
| Hepatocytes in | All hepatic enzymes | 1. ABT (general CYP | Hepatic metabolism with |
| suspension¹ | | inhibitor) | and without inhibitor |
| | | 2. Quinidine (CYP2D6 | |
| | | inhibitor) | |
| rhCYP2D6 | 2D6 | — | Study enzyme kinetics of |
| Supersomes^{1, 2} | | | CYP2D6 metabolism |

¹Human in vitro enzyme preparate of commercial origin.
²Supersomes = Recombinant enzymes prepared from baculovirus-transfected insect cells.

In vitro incubations in hepatocytes were performed as detailed in Table 2.

TABLE 2
Study conditions for studying intrinsic clearance of tasipimidine (1 μM)
in human hepatocyte incubation with and without inhibitors (n = 2).

| | Preparation | | | Inhibitor | | Incubation |
|---|---|---|---|---|---|---|
| | conc. (million | Pre- | | concentration | Inhibitor | time |
| Matrice | cells) | incubation | Inhibitor | (μM) | target | (min) |
| Suspended hepatocyte | 1 | No | — | — | — | 120 |
| Suspended hepatocyte | 1 | Yes | ABT | 1000 | CYPs | 120 |
| Suspended hepatocyte | 1 | No | Quinidine | 2 | CYP2D6 | 120 |
| Buffer | — | No | — | — | — | 120 |
| Buffer | — | Yes | — | — | — | 120 |

Enzyme kinetics of tasipidimine metabolism by rhCYP2D6 was studied by incubating the test compound at 0.16, 0.41, 1, 2.6, 6.4, 16, 40 and 100 μM concentrations containing 10 pmol/ml of the CYP enzyme for 0, 5, 10, 20, and 30 min. Enzyme kinetics was estimated from CLᵢₙₜ values measured for the test compound at different concentrations while metabolite characterisation was not performed.

The analysis for disappearance of tasipimidine and the identification of the formed metabolites were carried out by using a high-resolution liquid chromatographic-mass spectrometer supported by accurate masses (UPLC-HRMS). Firstly, metabolic stability of tasipimidine in different in vitro matrices was determined by monitoring the disappearance of the parent compound as a function of time. Moreover, structures of the formed metabolites were characterized from their respective product ion spectra. The synthetized reference compounds of the metabolites were used to confirm the identification of those specific metabolites.

### In Vitro Results

Tasipimidine is metabolised in human in vitro systems primarily via CYP enzymes, CYP2D6 in particular. This finding was supported by (I) recombinant CYP incubations and (II) hepatocyte incubations with general CYP inhibitor ABT and selective CYP2D6 inhibitor quinidine.

The measured intrinsic clearance (CLᵢₙₜ) of tasipimidine (1 μM) in rhCYP2D6 was 1.75 μl/min/pmol CYP and apparent enzyme kinetic parameters Km and Vmax 0.53 UM and 3.1 pmol/min/pmol CYP, respectively.

CYP1A1, CYP1A2, and CYP2C19 were identified as minor contributors to tasipimidine metabolism.

Following metabolic pathways were found in tasipimidine metabolizing preparates:
O-dealkylation to metabolite 1 and subsequent further oxidation (N— or O—) as well as dehydrogenation to metabolite 2 (catalysis by CYP2D6>>CYP1A1, CYP1A2, CYP2C19)
Oxidation (N— or O—), and dehydrogenation to metabolite 2 (catalysis by CYP2D6>>CYP2C19
CYP2D6 catalyzed formation of all the detected metabolites

In conclusion, CYP2D6 was identified as the major enzyme catalyzing tasipimidine metabolism in human.

### Example 2: First-In-Man Study

The first-in-man study was a randomized, double-blind, placebo-controlled, single-dose escalation study with healthy female and male subjects aged 18-44 years. The subjects were allocated to cohorts of 8 subjects of which 6 received active treatment and 2 received placebo. Each subject received either 1 dose of tasipimidine oral solution or 1 dose of placebo oral solution during the study. The studied doses of tasipimidine were 10, 25, 50, 100, and 150 μg as oral solution.

The subjects were genotyped for CYP2D6 on screening. The translation of CYP2D6 genotype to CYP2D6 phenotype was done according to the national consensus published by the Dutch Pharmacogenetics Working Group from KNMP (https://www.knmp.nl/index.php/media/113). The subjects with CYP2D6 poor metabolizer genotype were excluded from the study. The translation of CYP2D6 genotype to activity score was done according to the CYP2D6 Allele Functionality Table of PharmGKB (https://www.pharmgkb.org/page/cyp2d6RefMaterials).

Frequent blood samples were taken through an intravenous cannula for the assessment of the concentration of tasipimidine and its metabolite 1 in plasma. The sampling times were before (0 h) and 15 min, 30 min, 45 min, 1, 1.5, 2, 3, 4, 5, 6, 8, 10, 12, 16, and 24 hours after the study treatment administration. The PK parameters were calculated from the plasma concentration-time data by noncompartmental method using the commercial Phoenix WinNonlin software version 8.3.

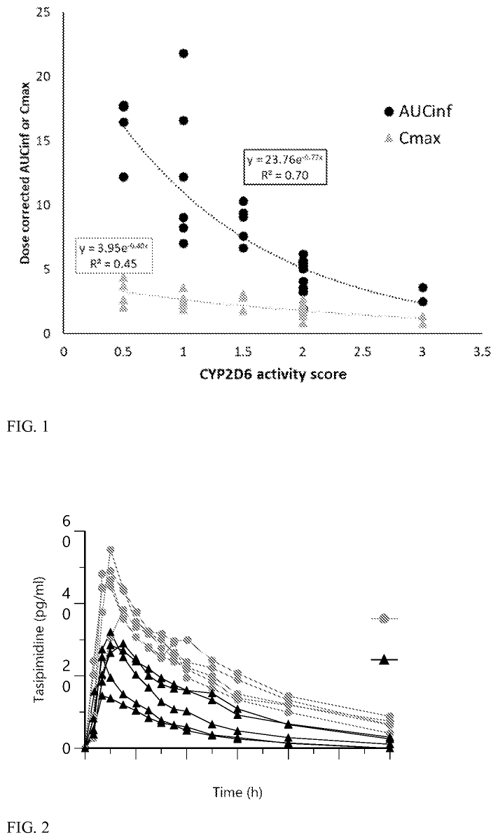

The pharmacokinetic data available from the single dose part of the study provides strong support for the role of CYP2D6 as the main elimination route for tasipimidine by showing correlation with tasipimidine AUCinf and Cmax with the CYP2D6 activity scores (FIG. 1). In addition, decreased variability (CV %) in AUCinf of tasipimidine after CYP2D6 activity score correction supports this finding (Table 3).

TABLE 3
Tasipimidine AUCinf [Mean (CV %), n = 6] from
the single dose part of the first-in-man study (AS, CYP2D6
genotype based activity score).

| Dose (μg) | AUCinf (h*pg/ml) | | AUCinf * AS (h*pg/ml) | |
|---|---|---|---|---|
| 10 | 52 | (46) | 90 | (34) |
| 25 | 186 | (61) | 254 | (15) |
| 50 | 360 | (71) | 475 | (40) |
| 100 | 1028 | (63) | 1026 | (12) |
| 150 | 1741 | (46) | 2033 | (39) |

### Example 3: PK Simulations Using the PBPK Model

A physiologically based pharmacokinetic (PBPK) model was used to simulate the plasma exposure of tasipimidine in fasted healthy 30 years old males of 70 kg with different CYP2D6 phenotypes. Simulations were produced with the commercial GastroPlus® 9.8.2 software. The input parameters of the model are presented in Table 4.

TABLE 4
The input parameter values of the PBPK model.

| Parameter | Value | |
|---|---|---|
| MW | 232.28 | g/mol |
| logP | 1.66 | |
| pKa | 9.24 | |
| Caco-2 Papp | 1.54 × 10⁻⁵ | cm/s |
| Blood to plasma ratio | 1.13 | |
| Fraction unbound in plasma | 86% | |
| Km for CYP2D6 | 0.106 | mg/l |
| Vmax for CYP2D6 | 0.000043 | mg/s |
| Intersystem extrapolation factor | 0.2 | |
| Renal Clearance | 6.3 | l/h |

PBPK simulations predicted significantly increased AUCinf, Cmax and t½ for tasipimidine in subject with CYP2D6 poor metabolizer phenotype (CYP2D6 activity=0) compared to subject with average CYP2D6 activity (Table 5).

TABLE 5
Simulated effect of CYP2D6 activity on the
PK parameters of 10 μg oral tasipimidine.

| CYP2D6 activity | AUCinf (h*pg/ml) | Cmax (pg/ml) | t½ (h) |
|---|---|---|---|
| Average | 69 | 17 | 3.4 |
| Poor metabolizer | 616 | 43 | 11.5 |
| Ratio poor/average | 8.9 | 2.5 | 3.4 |

### Example 4: Observed Effect of Paroxetine (a Strong CYP2D6 Inhibitor) Co-Treatment on the PK of Tasipimidine

Effect of co-treatment with a strong CYP2D6 inhibitor paroxetine on the PK of tasipimidine was studied. The study was an open label, crossover study with 5 healthy female and/or male subjects aged 26-51 years. The study started with a 8 days long once daily treatment of paroxetine 20 mg tablet. On the last day of paroxetine treatment, a single 10 μg dose of tasipimidine as oral solution was administered 1 h after the paroxetine intake after an overnight fast.

The subjects were genotyped for CYP2D6 on screening. The translation of CYP2D6 genotype to CYP2D6 phenotype was done according to the national consensus published by the Dutch Pharmacogenetics Working Group from KNMP (https://www.knmp.nl/index.php/media/113). The subjects with CYP2D6 poor metabolizer genotype were excluded from the study. The translation of CYP2D6 genotype to activity score was done according to the CYP2D6 Allele Functionality Table of PharmGKB (https://www.pharmgkb.org/page/cyp2d6RefMaterials).

Frequent blood samples were taken through an intravenous cannula for the assessment of the concentration of tasipimidine and its metabolite 1 in plasma. The sampling times were before (0 h) and 20 min, 40 min, 1, 1.5, 2, 2.5, 3, 3.5, 4, 5, 6, 8, 12, 24, 36, and 48 hours after the study treatment administration. The PK parameters were calculated from the plasma concentration-time data by noncompartmental method using the commercial Phoenix WinNonlin software version 8.3.

The AUCinf, Cmax and t1/2 of tasipimidine were significantly increased when paroxetine was co-administered (FIG. 2, Table 6).

TABLE 6
Mean (CV %) PK parameters of 10 μg oral tasipimidine
with and without paroxetine co-administration
(n = 5, mean CYP2D6 activity score = 1.2).

| | AUCinf (h*pg/ml) | | Cmax (pg/ml) | | t½ (h) | |
|---|---|---|---|---|---|---|
| Without paroxetine | 106 | (47) | 26 | (26) | 2.6 | (26) |
| With paroxetine | 281 | (14) | 47 | (13) | 4.3 | (15) |
| Ratio with/without | 3.1 | (44) | 1.9 | (23) | 1.7 | (13) |

A person skilled in the art will appreciate that the embodiments described herein can be modified without departing from the inventive concept. A person skilled in the art also understands that the present disclosure is not limited to the particular embodiments disclosed but is intended to also cover modifications of the embodiments that are within the scope of the present disclosure.

1. A combination comprising tasipimidine, or a pharmaceutically acceptable salt thereof, and a CYP2D6 inhibitor.

2-4. (canceled)

5. The combination according to claim 1, wherein the CYP2D6 inhibitor is a strong CYP2D6 inhibitor.

6. The combination according to claim 1, wherein the CYP2D6 inhibitor is paroxetine, bupropion, fluoxetine, quinidine, terbinafine, dacomitinib, pridopidine, tipranavir, or 3,4-methylenedioxymethamphetamine.

7. The combination according to claim 1, wherein the CYP2D6 inhibitor is paroxetine or bupropion.

8-9. (canceled)

10. A method of administering tasipimidine, wherein the method comprises administering tasipimidine or a pharmaceutically acceptable salt thereof, in combination with a CYP2D6 inhibitor, to a human being in need of treatment with tasipimidine.

11. A method for the treatment of a neuropsychiatric disorder, wherein the method comprises administering a therapeutically effective amount of tasipimidine or a pharmaceutically acceptable salt thereof, in combination with a CYP2D6 inhibitor, to a human being in need of treatment with tasipimidine.

12. The method according to claim 11, wherein the amount of the CYP2D6 inhibitor administered is sufficient to block tasipimidine metabolism.

13. The method according to claim 11, wherein the neuropsychiatric disorder is anxiety, depression, agitation, insomnia disorder, or insomnia disorder with comorbid depression, anxiety or pain.

14. The method according to claim 11, wherein the CYP2D6 inhibitor is a strong CYP2D6 inhibitor.

15. The method according to claim 14, wherein the CYP2D6 inhibitor is paroxetine, bupropion, fluoxetine, quinidine, terbinafine, dacomitinib, pridopidine, tipranavir, or 3,4-methylenedioxymethamphetamine.

16. A pharmaceutical composition comprising the combination according to claim 1 and at least one of a pharmaceutically acceptable excipient, a pharmaceutically acceptable carrier, or combinations thereof.

17. A method for the treatment of a neuropsychiatric disorder, wherein the method comprises administering the pharmaceutical composition according to claim 16 to a human being in need thereof.

18. The method according to claim 10, wherein the CYP2D6 inhibitor is a strong CYP2D6 inhibitor.

19. The method according to claim 18, wherein the CYP2D6 inhibitor is paroxetine, bupropion, fluoxetine, quinidine, terbinafine, dacomitinib, pridopidine, tipranavir, or 3,4-methylenedioxymethamphetamine.

20. The method according to claim 10, wherein tasipimidine plasma levels of the human being are increased.
